(19) United States
(12) Patent Application Publication
(10) Pub. No.: US 2026/0221291 A1
(43) Pub. Date: Jul. 30, 2026

(54) ARTIFICIAL INTELLIGENCE-BASED METHOD AND SYSTEM FOR PREDICTING MUTATION TRAJECTORIES OF PATHOGEN EVOLUTION

(71) Applicant: NEC Laboratories Europe GmbH, Heidelberg (DE)

(72) Inventors: Daniel ONORO-RUBIO, Heidelberg (DE); Raman SIARHEYEU, Heidelberg (DE)

(21) Appl. No.: 19/148,323

(22) PCT Filed: Jul. 4, 2023
(86) PCT No.: PCT/IB2023/056919

§ 371 Date: Jul. 16, 2025

Related U.S. Application Data
(60) Provisional application No. 63/496,420, filed on Apr. 17, 2023.

(51) Int. Cl. G16H 50/50

(52) U.S. Cl. CPC G16H 50/50

(57) ABSTRACT

A computer-implemented method for predicting pathogen evolution includes computing a mutation trajectory of an input sample of a pathogen to be simulated by iteratively determining an update to a vector of changes based on a gradient that is computed with respect to the input sample using a loss associated with a prediction output of a trained differentiable surrogate model. Then, the method includes reconstructing changes to the input sample based on the iterative updates to obtain a predicted pathway of the pathogen evolution. The present invention can be used in a variety of applications including, but not limited to, several anticipated use cases in medical diagnostics/applications and in healthcare, to improve machine learning, optimize processes or predictions or support decision making.

### CROSS-REFERENCE TO PRIOR APPLICATIONS

This application is a U.S. National Phase application under 35 U.S.C. § 371 of International Application No. PCT/IB2023/056919, filed on Jul. 4, 2023, and claims benefit to U.S. Patent Application No. 63/496,420, filed on Apr. 17, 2023. The International Application was published in English on Oct. 24, 2024 as WO 2024/218557 A1 under PCT Article 21 (2).

### FIELD

The present invention relates to artificial intelligence (AI) and machine learning, and in particular to a method, system and computer-readable medium for predicting mutation trajectories of pathogen evolution with applications to vaccine design.

### BACKGROUND

Mutations in viral pathogens are driven by evolutionary forces that allow them to constantly adapt to changing environmental conditions. This poses significant challenges in vaccine development and drug design since the designed substances lose their targets and efficacy. Moreover, mutations in wild-type viruses, combined with a favorable environment, can lead to interspecies transition and cause new dangerous infections in the human population. There are a number of currently known diseases such as the Zika, Ebola, SARS, MERS, and COVID-19 that are caused by viruses transmitted from animals to humans.

In general, mutations are considered unpredictable because of multiple known and unknown factors, and the occurrence of a mutation is usually considered as a random event. However, several methods have been proposed that try to predict regions in the viral genome that are most likely to mutate or even try to score the pathogenicity of viruses depending on how likely they are to jump to the human population.

Classical approaches predict the mutability along the viral proteome using conservation profiles built from multiple sequence alignments (MSA) of the related viruses (see Nagar, A., et al., “Fast discovery and visualization of conserved regions in DNA sequences using quasi-alignment,” BMC Bioinformatics 14 Suppl 11: S2 (September 2013), which is hereby incorporated by reference herein). On top of MSA, the classical approaches calculate genome conservation features, such as site-based Shannon's entropy, or identify genome loci subject to strong selective constraints. The resulting models, however, have a number of technical limitations in that they normally require the alignment of an extensive number of sequences collected over time, have few parameters and have limited predictive power.

Rodriguez-Rivas, J., et al., “Epistatic models predict mutable sites in SARS-COV-2 proteins and epitopes,” PNAS 119(4): e2113118119 (January 2022), which is hereby incorporated by reference herein propose a method that uses data from coronaviruses, preexisting to SARS-COV-2, to build alignments of homologous sequences and train statistical sequence models to predict the mutability of each position. It also tries to account for complex patterns resulting from epistasis while assigning mutability scores. The inference of the model parameters that consider high-order epistatic interactions is computationally hard.

There also have been attempts to identify viral mutations that have potential to spread in the population based on features from viral epidemiology, evolution, immunology, and neural network-based protein sequence modeling. However, it is not obvious and is technically challenging to determine which types of features or combinations of features will have the best predictive power. Yan, S., et al., “Application of neural network to predict mutations in proteins from influenza A viruses-A review of our approaches with implication for predicting mutations in coronaviruses,” J. Phys. Conf. Ser. 1682(1): 012019 (November 2020), which is hereby incorporated by reference herein propose a method that adopts the ideas of statistical physics to calculate mutation-based features from MSA of viral proteins and applies a neural network to predict the probabilities of mutations in influenza A virus genes. According to Maher, M., et al., “Predicting the mutational drivers of future SARS-COV-2 variants of concern,” Sci Transl Med. 14(633) (February 2022), which is hereby incorporated by reference herein, the highest predictive power of a model was obtained from an epidemiological feature, namely, the exponentially weighted mean ranking across epidemiological variables (mutation frequency, fraction of unique haplotypes in which the mutation occurs, and the number of countries in which it occurs), which was validated by predicting driver mutations in emerging SARS-COV-2 variants of concern.

Grange, Z., et al., “Ranking the risk of animal-to-human spill-over for newly discovered viruses,” PNAS 118(15): e2002324118 (April 2021), which is hereby incorporated by reference herein, estimate a risk score for viruses originating in wildlife for animal-to-human transmission by weighting and averaging risk factors, identified from literature reviews and input from experts. Text-mining techniques can also be applied to viral genomes to estimate the mutability of genomic segments. Such methods may rely on calculating the importance of genomic segments based on their spatial distribution and frequency over the whole genome (see Darooneh, A., et al., “A novel statistical method predicts mutability of the genomic segments of the SARS-COV-2 virus,” QRB Discovery 3, E1 (December 2021), which is hereby incorporated by reference herein), which is similar to the keyword detection techniques in text-mining.

A state-of-the-art model referred to as PyR0 uses a hierarchical Bayesian multinomial logistic regression that infers relative prevalence of viral lineages across geographic regions and detects lineages increasing in prevalence to identify mutations relevant to fitness as a part of a downstream feature selection procedure (see Obermeyer, F., et al., “Analysis of 6.4 million SARS-COV-2 genomes identifies mutations associated with fitness,” Science 376(6599:1327-1332 (June 2022), which is hereby incorporated by reference herein). The model was able to determine which mutations were becoming more common and estimate how quickly each mutation could cause the SARS-COV-2 lineages to spread. Stern, A., et al., “The Evolutionary Pathway to Virulence of an RNA Virus,” Cell 169 (1): 35-46.e19 (March 2017), which is hereby incorporated by reference herein, propose a Markov model to analyze viral genomes assuming that natural selection would lead to an increase in the rate of substitutions into certain nucleotides and decrease the loss of those nucleotides in some loci. The model was used to find genome sites under selection pressure, reconstruct mutation events and mutation trajectories that lead attenuated poliovirus to evolve into virulent strains in human population. However, those methods can only operate with the mutation events that have been already observed and do not have the technical capability to score mutations that are not yet in population.

### SUMMARY

In an embodiment, the present invention provides a computer-implemented method for predicting pathogen evolution. The method includes computing a mutation trajectory of an input sample of a pathogen to be simulated by iteratively determining an update to a vector of changes based on a gradient that is computed with respect to the input sample using a loss associated with a prediction output of a trained differentiable surrogate model. Then, the method includes reconstructing changes to the input sample based on the iterative updates to obtain a predicted pathway of the pathogen evolution. The present invention can be used in a variety of applications including, but not limited to, several anticipated use cases in medical diagnostics/applications and in healthcare, to improve machine learning, optimize processes or predictions or support decision making.

### BRIEF DESCRIPTION OF THE DRAWINGS

Embodiments of the present invention will be described in even greater detail below based on the exemplary figures. The present invention is not limited to the exemplary embodiments. All features described and/or illustrated herein can be used alone or combined in different combinations in embodiments of the present invention. The features and advantages of various embodiments of the present invention will become apparent by reading the following detailed description with reference to the attached drawings which illustrate the following:

FIG. 1 is a flow diagram of a method to create a surrogate model according to an embodiment of the present invention;

FIG. 2 is a schematic diagram illustrating a surrogate model design according to an embodiment of the present invention;

FIG. 3 is a flow diagram schematically illustrating a method according to an embodiment of the present invention;

FIG. 4 is a schematic diagram illustrating the surrogate model and how the updates are performed according to an embodiment of the present invention; and

FIG. 5 is a block diagram of an exemplary processing system, which can be configured to perform any and all operations disclosed herein.

### DETAILED DESCRIPTION

Embodiments of the present invention provide an AI-based method and system to predict possible mutation trajectories of viral evolution that could lead to its transition from animals to humans. The method introduces changes to the viral genome and estimates the chances that the changes would lead to a host transition or high increase in viral fitness. Embodiments of the present invention advantageously enhance computation functionality of AI systems to enable to point to the regions in viral genome that are prone to drive potential “gatekeeper” mutations, which has applications, for example, to improve vaccine design.

Embodiments of the present invention address and provide solutions to the technical problem of how to accurately predict and score possible genome changes in viral pathogens that are needed for a pathogen to enter the human population or significantly increase its fitness in human population. The AI-based method according to embodiments of the present invention utilizes adversarial attacks that iteratively introduce small, biologically meaningful modifications into a viral genome that will eventually lead to a host transition or contribute to continuous increase in the viral fitness. Embodiments of the present invention enable to identify the regions in the viral genome where important mutations are most likely to occur, as well as to generate potential evolutionary trajectories. As mentioned above, embodiments of the present invention can be practically applied to effect improvements in the field of vaccine design.

In particular, embodiments of the present invention can be used as a computational tool in vaccine design with enhanced functionality to guide the creation of new vaccines based on the prediction of the minimal changes in the genome of an existing virus that currently only affects animals, such that it can start infecting humans. According to an embodiment of the present invention, a virus is chosen from the animal kingdom and modifications are iteratively added to its genome following the gradient of a derivable surrogate model.

Embodiments of the present invention can be practically applied to predict or explain evolution of a protein from a protein version with properties A into a protein version with properties B. To do this, examples of both versions of the protein are included in training dataset. The predictions could include a prediction of how a virus may evolve to be more/less fit for different environments (e.g., climates) and/or a prediction of how quickly a virus may evolve to be more fit to certain human genome traits versus others (e.g., predicting what groups or individuals the virus will be most likely to infect). The predictions can be used to identify regions in the viral genome where important adaptation mutation are likely to occur and/or to predict how the sequence of the next mutated/more adopted virus variant may look like, which advantageously provide for early design of a vaccine against next most likely virus variants.

According to a first aspect, the present invention provides computer-implemented method for predicting pathogen evolution includes computing a mutation trajectory of an input sample of a pathogen to be simulated by iteratively determining an update to a vector of changes based on a gradient that is computed with respect to the input sample using a loss associated with a prediction output of a trained differentiable surrogate model. Then, the method includes reconstructing changes to the input sample based on the iterative updates to obtain a predicted pathway of the pathogen evolution.

According to a second aspect, the present invention provides the computer-implemented method according to the first aspect, wherein the updates are determined iteratively until a stop criteria is met, after which the step of reconstructing the changes to the input sample is performed.

According to a third aspect, the present invention provides the computer-implemented method according to the first or second aspect, wherein the stop criteria is based on a predetermined number of iterations or changes to the input sample, or is based on the prediction output of the surrogate model.

According to a fourth aspect, the present invention provides the computer-implemented method according to any of the first to third aspects, wherein the surrogate model comprises an encoder, a decoder and a classifier, and is trained by: inputting training samples and corresponding labels as inputs to the encoder that converts the inputs into latent representations; inputting the latent representations into the decoder that reconstructs the inputs; determining a reconstruction loss associated with the reconstructed inputs; inputting the latent representations into the classifier that provides a classification of each of the latent representations to fit the labels; determining a classification loss associated with the classifications; and training the surrogate model using the reconstruction loss and the classification loss.

According to a fifth aspect, the present invention provides the computer-implemented method according to any of the first to fourth aspects, wherein, at each iteration, the update to the vector of changes is added to a latent representation of the input sample and used for a subsequent prediction of the surrogate model, from which a subsequent loss is determined for determining a subsequent gradient for a subsequent update.

According to a sixth aspect, the present invention provides the computer-implemented method according to any of the first to fifth aspects, wherein the changes to the input sample are reconstructed based on the latent representations used for the subsequent predictions of the surrogate model.

According to a seventh aspect, the present invention provides the computer-implemented method according to any of the first to sixth aspects, wherein the updates to the vector of changes take into account an auxiliary loss term indicating whether the changes to the input sample are plausible mutations.

According to an eighth aspect, the present invention provides the computer-implemented method according to any of the first to seventh aspects, further comprising storing the updates to the vector of changes, and tracking a path of the changes to the input sample based on the stored updates to the vector of changes.

According to a ninth aspect, the present invention provides the computer-implemented method according to any of the first to eighth aspects, further comprising collecting a dataset that contains examples of viruses that infect humans, and viruses that infect animals, and training the surrogate model using the dataset as training data to at least classify whether a virus would infect a human or an animal as the prediction.

According to a tenth aspect, the present invention provides the computer-implemented method according to any of the first to ninth aspects, further comprising collecting a dataset that contains genome sequences on viruses, labels that distinguish between animal and human version of the viruses and/or fitness information on the viruses, and training the surrogate model using the dataset as training data to predict viral fitness, wherein the loss is a regression loss, and wherein changes to the input sample identify antigen targets.

According to an eleventh aspect, the present invention provides the computer-implemented method according to any of the first to tenth aspects, further comprising designing a vaccine based on the predicted pathway of the pathogen evolution.

According to a twelfth aspect, the present invention provides the computer-implemented method according to any of the first to eleventh aspects, wherein the changes to the input sample are used to rank potential variants of the pathogen, and/or to determine convergently evolving features and/or patterns in a genome of the pathogen that make humans susceptible to the pathogen.

According to a thirteenth aspect, the present invention provides the computer-implemented method according to any of the first to twelfth aspects, further comprising collecting a dataset that contains annotated genome sequences of bacterial pathogens and labels distinguishing susceptible and antibiotic-resistant strains of the bacterial pathogens, and training the surrogate model using the dataset as training data, wherein the changes to the input sample indicate genome regions and homologous proteins susceptible to development of antibiotic resistance.

According to a fourteenth aspect, the present invention provides a computer system comprising one or more hardware processors which, alone or in combination, are configured to perform the method according to any of the first to thirteenth aspects.

According to a fifteenth aspect, the present invention provides a tangible, non-transitory computer-readable medium having instructions thereon which, upon being executed by one or more processors, cause execution of the method according to any of the first to thirteenth aspects.

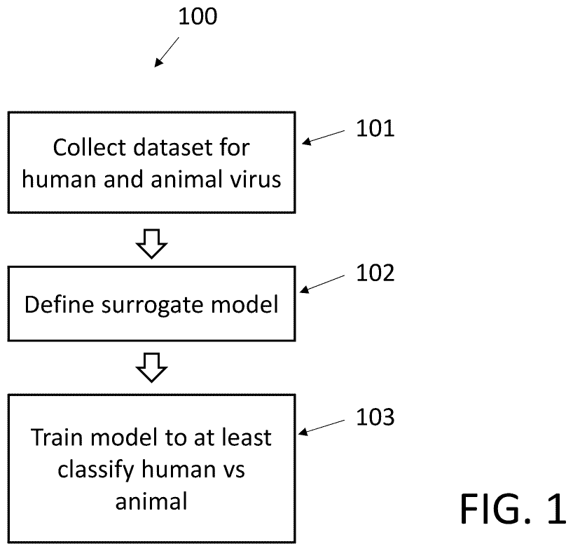

According to an embodiment of the AI-based method according to an embodiment of the present invention, the first step is the creation of a surrogate model. FIG. 1 illustrates the steps of a method 100 for training the surrogate model. This process starts with the dataset acquisition and preparation in a first step 101. This step 101 may involve adapting existing datasets or collecting new samples. As a result, a dataset is obtained that at least contains animal and human virus samples. Additional information can also be collected, and can be used as an extra supervision during the training or during the inference (for example, adding labels that indicate whether a virus can infect, but also can cause a disease). The second step 102 is defining a surrogate model. The surrogate model can be any differentiable model that can efficiently fit the training data. The third step 103 is to train the surrogate model to fit the training data, at least to be able to classify as human or animal.

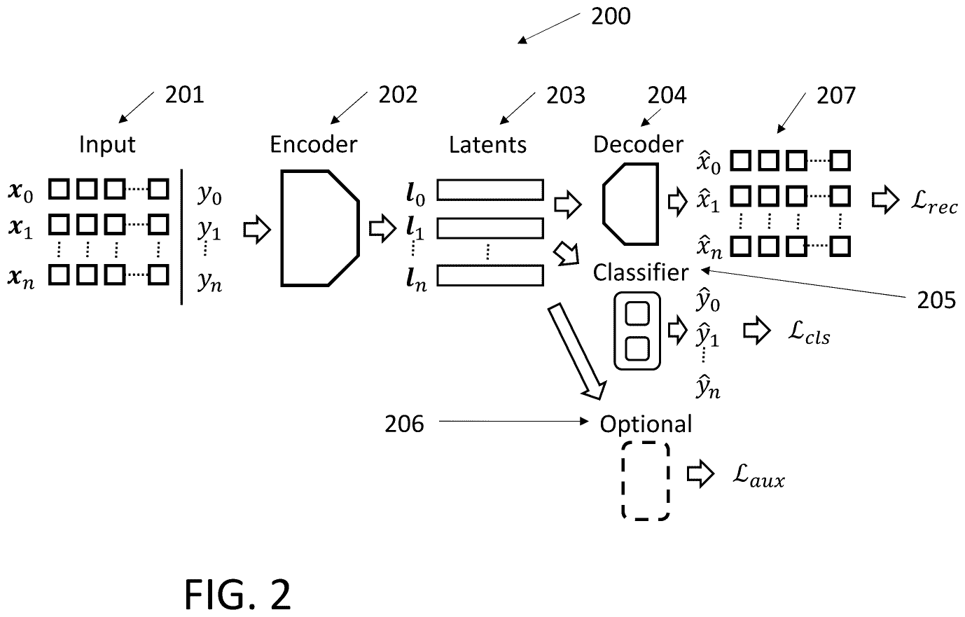

FIG. 2 illustrates a schematic diagram of an exemplary surrogate model 200 according to an embodiment of the present invention. In the first column is the input 201 comprising a sequence or set of inputs xᵢ (x₀, x₁ . . . xₙ) of the surrogate model 200 with their corresponding labels yᵢ (y₀, y₁ . . . yₙ). The inputs can be, in particular, strings of nucleotides (in case of DNA), ribonucleotides (in case of RNA) or amino acids (in case of consideration of only viral proteins) obtained from a virus. For example, xᵢ can correspond to a sequence of amino acids (in case of consideration of a protein of interest) that is extracted from virus_i. Labels here can include ‘animal’ or ‘human’ versions of virus_i. Additional possible labels in different cases can include: ‘adopted’ or ‘not adopted’; ‘dangerous’ or ‘not dangerous’; ‘low infectious’ or ‘high infectious’, etc. The data is fed into an encoder 202 that converts the input 201 into latents 203 comprising latent representations lᵢ (l₀, l₁ . . . lₙ). Latents are used by models such as deep neural networks to encode the information as a set of features that they learn to make predictions. The latent representations lᵢ can then be used in two branches. The top branch contains a decoder 204. The decoder 204 takes the latent representations lᵢ and tries to reconstruct the inputs xᵢ to output reconstructed inputs \hat{x}ₗ ( [symbol] , [symbol] . . . . [symbol] ). This branch is connected to a reconstruction loss [symbol] _{rec} that will be used during model training. The lower branch is a classification branch containing a classifier 205 which takes the latent representations lᵢ and classifies them to fit the labels yᵢ to produce the output ŷₗ ( [symbol] , [symbol] . . . [symbol] ) as the classification. This branch is connected to a classification loss [symbol] _{cls} that will be used both during training and inference. Finally, in certain embodiments, additional branches 206 can be added for extra supervision. For example, additional branch 206 may contain a loss that is calculated based on the probability that the reconstructed viral sequence obtained in the current iteration is not biologically meaningful or may not exist. As another example, additional branch 206 could provide a classification of whether a virus, in addition to infecting, human can cause a certain disease, and a corresponding classification loss. Thus, in this case there would be added an additional output to classify the disease, with its corresponding classification loss (for example, cross entropy loss).

Once the Surrogate Model 200 is Trained, the Mutations Required for an Animal Virus to Infect a Human are Calculated. For Example, the Training of the Differentiable Surrogate Model 200 could be Performed in Accordance with the Following Pseudocode:
for epoch in N_EPOCHS:
   for batch in data:
      X, y=batch
      pred=model (X)
      l=loss_func (pred, y)
      grad=compute_gradients (model, l)
      update_model (model, grads)

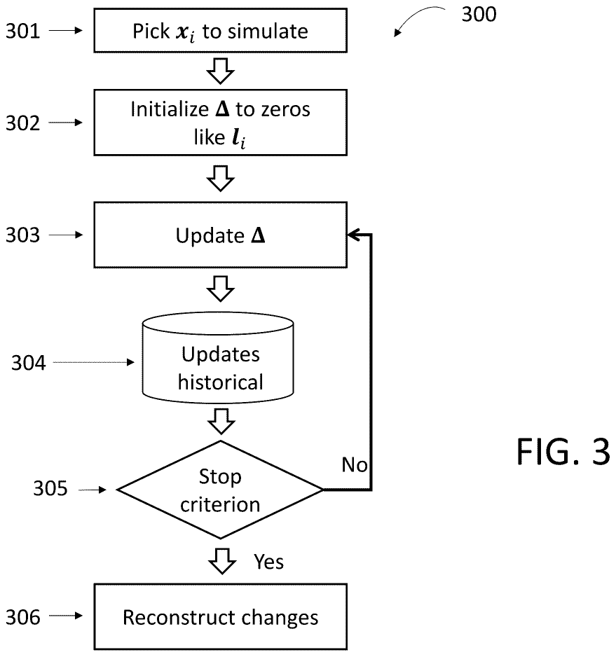

FIG. 3 depicts a high-level diagram of an iterative update process 300 according to an embodiment of the present invention. The first step 301 is to pick the animal virus sample xᵢ of interest. Then, in a second step 302, a vector of changes Δ, representing changes to be added, is initialized. At the start, the vector of changes Δ is initialized with zeros. For example, where the latent has two components (lᵢ=[0.5,1.2]), then vector of changes Δ=[0,0]. Next, in a third step 303, the vector of changes Δ is updated, and the updated vector of changes Δ is stored in a database containing a history of updates in a fourth step 304. In a fifth step 305, it is checked whether a stop criterion has been met, and if not, the process iterated back to the third step 303. Once the stop criterion has been met, the changes are reconstructed in a sixth step 306.

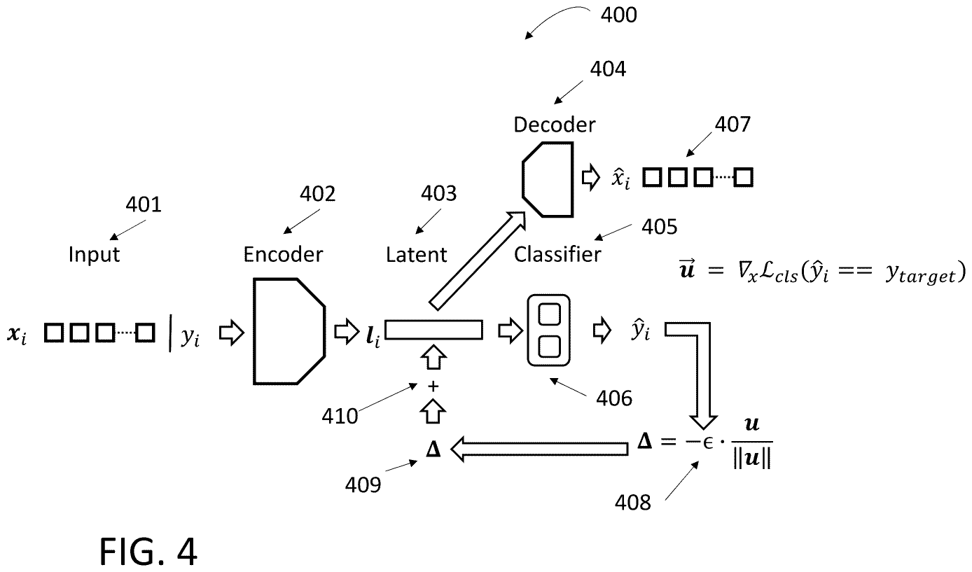

FIG. 4 illustrates a more detailed diagram of an update procedure 400 showing how the updates are performed in the third step 303. The selected sample xᵢ is passed as input 401 to the encoder 402 and it produces the latent 403 as a latent vector lᵢ. This latent vector lᵢ is now passed to the classification branch, which will use the classifier 405 (e.g., the classifier 205 of FIG. 2 that has been trained) to at least classify it as a human or an animal virus as output ŷₗ. Then, updates u are computed in a step 406 by computing the gradient with respect to the input sample xᵢ as follows:

$$u = {∇}_{l} ( ℒ ) ,$$

where ∇ₗ denotes the gradient of the loss function [symbol] . The loss function [symbol] should at least contain cross-entropy classification loss [symbol] _{cls} and it can be optionally extended with other auxiliary loss [symbol] ₐᵤₓ. For example, in some embodiments where there is an auxiliary loss [symbol] ₐᵤₓ, the loss can be the cross-entropy classification loss [symbol] _{cls} or a sum of the cross-entropy classification loss [symbol] _{cls} and the auxiliary loss [symbol] ₐᵤₓ. Thus, guided by the trained (or pre-trained) surrogate model, modifications to the input sample xᵢ are determined which result in the surrogate model starting to classify the sample as a human virus rather than an animal virus.

Accordingly, embodiments of the present invention provide the functionality to generate updates u over the given input sample xᵢ such that the surrogate model shifts its prediction, similarly as it is done in adversarial attacks. The auxiliary loss [symbol] ₐᵤₓ is present in some, but not all embodiments, and can be a single loss or the sum of many losses. Examples include a penalty on gene transitions that are known to not occur, the prediction of valid vs. invalid virus sequences, the classification loss of a virus causing a particular disease, etc. The auxiliary loss [symbol] ₐᵤₓ can be obtained from a third-party model that is trained to predict, for example, a protein structure from its amino acid sequence and a likelihood that this version of the protein is stable enough and/or a likelihood that this version of the protein may bind to a corresponding receptor on a surface of target cells. The likelihood can be converted into a loss term in multiple ways (e.g. by taking a logarithm of the likelihood) and depends on the score that the third-party model provides. In a simple case of DNA/RNA input sequences, it would be possible to try to translate a sequence into a protein and assign high losses in case the sequence can't be correctly translated into a protein (e.g., it contains a stop-codon in the middle). Another example could be to use an additional loss term to penalize transitions that lead to impossible changes. For example, if it is known that particular gene mutations cannot take place, those transitions are classified, and guides the method towards a change that is possible to be taken by the virus.

Once the updates u have been computed in step 406, the updates u are normalized in a step 408 by dividing by the modulo and multiplying them by a predefined hyper-parameter e that is fine-tuned as follows:

$$Δ = - ϵ \cdot \frac{u}{‖ u ‖}$$

The hyper-parameter e acts as a learning rate. When updates are made following the gradients, this typically will create very large updates that can make the process unstable, and thus an easy and efficient way to address this is to multiply the gradients by a small value (for example 0.01).

After the vector of changes Δ is computed at step 410, it is added to the latent vector lᵢ at step 411. Referring again now also to FIG. 3, the updates u are then stored (for example, they can be useful for tracking the path of changes that the virus might take) in a database in the fourth step 304, and new updates are computed. The update procedure 400 is repeated until the stop criterion is met in the fifth step 305 (for example, the maximum number of iterations, the number of introduced changes, whether the surrogate model classifies the virus as capable of infecting humans, etc.). Once the stop criterion is met, the changes in the inputs are reconstructed from the resulting latent representations to see the changes in the viral genome in the sixth step 306 (reconstruction=decoder(lᵢ)). From that point on, the mutations that the virus needs to incorporate into its genome so that it can infect humans are obtained from the output 407 of the decoder 404, which provides a single reconstructed structure of the input sample xᵢ. Thus, the decoder 404 is used once the iterative update process is completed to obtain the new predicted structure. Finally, this information can be used to synthesize new vaccines that can target new virus strains or species.

Embodiments of the present invention can be practically applied to effect improvements in technical fields such as vaccine design, AI drug development or personalized medicine. For example, an embodiment of the present invention can be used to identify antigen candidates (hotspots) for designing antiviral vaccines with respect to virus evolution, and to accelerate the development of vaccines against epidemic and pandemic threats. In particular, embodiments of the present invention can predict and select hotspots for antiviral vaccines that account for potential/putative future virus variants of concern. Here, a “hotspot” refers to an immunogenic part of a viral protein. Thus, when a hotspot or a part of it is demonstrated to the human immune system, it is likely to cause an immune response against the virus. This enables more effective vaccine design, for example, making a ‘mutant-proof’ vaccine against a broad range of beta-coronaviruses and accounting for potential new high-risk variants as an enhancement of existing hotspot selection and optimization pipelines. As inputs, the data source can include: genome sequence database on SARS-COV-2 and other beta-coronaviruses; labels that distinguish between animal and human versions of the considered virus sequences; and/or virus fitness information (for example, incidence, prevalence, and infectivity of the virus variants in human population) and metadata. Application of the method according to an embodiment of the present invention provides to simulate hypothetical evolution of a pathogen such that it follows the peaks (best fit) in the host/environmental fitness landscape and analyze mutations. The receptor-binding domain is normally considered as a good source for a vaccine antigen because it could induce neutralizing antibodies that prevent host cell attachment and infection. The AI-based method according to an embodiment of the present invention can be used to simulate evolutionary trajectories of spike proteins(S) of beta-coronaviruses, particularly the receptor-binding domain in following scenarios:
1) Training on data from beta-coronaviruses that passed animal-to-human transition, and using the trained model to predict potential mutations that might allow such a transition for other beta-coronaviruses with a high spill-over risk.
2) Training on historical SARS-COV-2 data collected during the COVID pandemic and using the trained model to predict the emergence of future variants of concern for currently circulating SARS-COV-2 virus strains. To do this, in the proposed surrogate model (see FIG. 2) the classification task is transformed into a regression task: the variables y and ŷ will denote the true and predicted viral fitness, and instead of classification loss [symbol] _{cls}, a regression loss [symbol] _{reg} will be used.

Structural characteristics of viral proteins will be accounted in the surrogate model via loss [symbol] ₐᵤₓ (see FIG. 2) to prevent mutation trajectories that would make the viral proteins non-functional and the virus non-viable. The output could be a list of evolved genomic sequences that will be used for further hotspot selection and vaccine construction, a list of important mutations in viral proteins that reveal common patterns of potential virus evolution and/or most conserved genome regions/positions. As automated decisions or actions (technicity), the finally predicted sequences and the effective hotspots derived from them can be sent for lab examination and experimentations to construct a vaccine with coverage against likely future virus variants of concern.

An embodiment of the present invention could also be practically applied to explore scenarios for the emergence of antibiotic resistance to support decision-making on optimization of individual therapy, for example, modeling possible scenarios of antibiotic resistance in bacterial pathogens based on their genome sequence data. Understanding how close a pathogen is to turning into an antibiotic resistant strain, in terms of anticipated mutation efforts, will provide valuable feedback for optimizing therapy to avoid adverse disease scenarios. As inputs, the data source can include: a database of annotated genome sequences of bacterial pathogens; labels that distinguish between susceptible and antibiotic-resistant strains of the pathogen for the considered drugs (surrogate model trained to output a classification of “susceptible” or “resistant” for the input sample bacteria with respect to a particular drug); and/or sequences data of the pathogens for the patients in question. Application of the method according to an embodiment of the present invention provides to train the surrogate model on the genome regions and sets of homologous proteins that are involved in the development of antibiotic resistance, and use the trained model to simulate hypothetical evolution of a susceptible pathogen such that it will be consistently classified as resistant and analyze suggested mutation trajectories. Accordingly, in this embodiment, the surrogate model is trained first to distinguish between drug-susceptible and drug-resistant genome sequences. Then, application of the method according to an embodiment of the present invention can be used to predict potential mutation pathways/trajectories that would lead to a drug-resistant version of an exposed bacterial genome. In other words, mutations are iteratively predicted, guided by the surrogate model, that may finally convert a microbe into a drug-resistant version. The output could be a list of mutations in the pathogen genome needed to turn a pathogen into a drug resistance strain, and/or the lengths of simulated mutation trajectories. For example, timing can be approximated based on the number of iterations done to convert a pathogen from drug-susceptible into drug-resistant, since one or more mutations are introduced at each iteration. The greater the number of iterations, the longer the mutation trajectories will take. As automated decisions or actions (technicity), the produced output would allow medical experts to take decisions on individual treatment strategies for patients considering the risk of developing drug resistance (for example, for tuberculosis therapy). Another decision support system could be running on top of the AI system according to an embodiment of the present invention to identify consistent treatment options.

An embodiment of the present invention could also be practically applied to estimate spill-over/antigenic shift risks for the viruses that do not circulate yet in human population. This use case addresses that humans consume or interact with animals and other species throughout their lifetime, which can cause transition of diseases or infections. A particular virus generally infects a certain type of cell and binds to a specific receptor when attacking a cell which ideally should be present in cells of different species. From viral and human genome sequences, application of the method according to an embodiment of the present invention provides to predict the probability that an animal-infecting virus will infect humans given biologically relevant exposure (spill-over potential). As inputs, the data source can include: large sequence datasets and metadata on viruses that had previously been assessed for human infection abilities based on published reports; datasets from big global efforts, such as the Global Virome Project; and/or sequences and structures of putative human receptors binding which might become an entry point for the virus into a human organism. In this use case, the surrogate model is trained on sequences of homologous genes extracted from virus genomes of human-infecting and non-human viruses explored due to large-scale infectious-diseases pan-genome analysis. Putative receptor data can be used to refine the model and prevent mutation trajectories that are destructive for virus proteins. The trained model is then applied to explore the potential mutational efforts needed for a virus to start infecting humans and use them to calculate genome-based spill-over risks. The output can be a ranked list of viruses with the assigned genome-based spill-over potential, and/or modelled mutation pathways. As automated decisions or actions (technicity), the AI system according to an embodiment of the present invention can be a computational tool integrated into a part of a workflow for proactive virus surveillance, for example:
1) Calculated genome-based spill-over risks are used in ranking the variants of concern and identification of candidate zoonoses with conditions for virus transition into the human population. This improves the forecasting abilities where these viruses may emerge.
2) Analyses of the suggested mutation pathways with another AI system that runs on top of the AI system according to an embodiment of the present invention to reveal the convergently evolving features or generalizable patterns in viral genomes that may preadapt viruses to infect humans. This would allow adjusting vaccine development strategies.

According to an embodiment of the present invention, new mutations are introduced iteration-by-iteration. This process is guided by the surrogate model that was trained to classify the ability of virus versions to infect humans (e.g. least likely against most likely). It is not needed for the model to directly assign the scores to the mutations. Rather, it is possible to do a number of simulations, analyze the predicted mutation pathways and calculate statistics on the mutations (e.g., in which parts of a virus genome do the mutations occur, and which mutations occurred most frequently).

Embodiments of the present invention provide for the following enhanced computer functionality and improvements over exiting technology:
1) The ability to compute mutation changes guided by the proposed loss that accounts for plausible mutation transitions, and/or biological validity of sequences, in an iterative procedure.
2) As it is observed from literature, existing technology focuses on identifying conserved regions by comparing pathogen genomes or ranking mutations that have been already observed in the existing virus variants (see Nagar, A, et al.; Rodriguez-Rivas, J., et al.; Yan, S., et al.; and Maher, M., et al.). In contrast, embodiments of the present invention enable to predict beneficial mutations that may happen, but haven't happened yet. This computationally challenging task is addressed using the AI-based method according to an embodiment of the present invention allowing to relatively quickly explore a complex genotype space and generate plausible mutation pathways that would adopt a microbe to a changing host-induced fitness landscape.
3) Existing technology predicts variants of concern among SARS-COV-2 lineages and identifies scenarios of spreading mutations, but only includes functionality for the mutations that have been observed in population (see Obermeyer, F., et al.). In contrast, embodiments of the present invention enable to forecast mutations that would increase fitness or allow the virus to infect humans even if they were not previously observed. Existing technology can only in some cases reconstruct mutation pathways and identify most important mutations related to virus adaptation, and can do this only for the historical data (see Stern, A., et al.).

In an embodiment, the present invention provides a method for predicting the changes that a given virus would have to take such that it can infect humans, the method comprising the steps of:
1) Collection of a dataset that contains examples of viruses that infect humans, and viruses that infect animals.
2) Training of a surrogate model, or receiving an already trained differentiable surrogate model, that can at least classify whether a virus would infect a human or an animal.
3) Selecting a sample of a virus of interest to simulate.
4) Computing the pathways that correspond to the evolution of the virus by:
   a. Running the gradient-based strategy that computes the closest mutation of a given virus based on the surrogate model.
   b. Storing the generated updates that the virus could take toward turning into a version that can infect humans.
   c. Using a stop criterion based on the surrogate model's decision shift, a convergence criterion, several iterations, or other heuristic.
5) Converting back the changes in the original format (for example, gene mutations) to obtain the whole mutation pathway (see output 407 of the decoder 404 in FIG. 4). This step converts the latent representation into the original format (e.g., DNA/RNA or amino acid sequence). The final result is obtained as the decoder output once the stopping criteria has been met (virus sequence with all final mutations) and all updates have been made. It is also possible to obtain the converted output from the latent space on every iteration, which would enable to restore the whole mutation pathway.

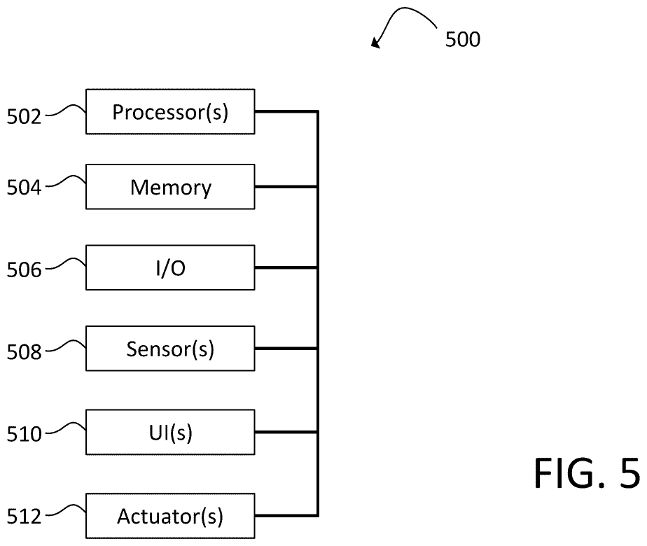

Referring to FIG. 5, a processing system 500 can include one or more processors 502, memory 504, one or more input/output devices 506, one or more sensors 508, one or more user interfaces 510, and one or more actuators 512. Processing system 500 can be representative of each computing system disclosed herein.

Processors 502 can include one or more distinct processors, each having one or more cores. Each of the distinct processors can have the same or different structure. Processors 502 can include one or more central processing units (CPUs), one or more graphics processing units (GPUs), circuitry (e.g., application specific integrated circuits (ASICs)), digital signal processors (DSPs), and the like. Processors 502 can be mounted to a common substrate or to multiple different substrates.

Processors 502 are configured to perform a certain function, method, or operation (e.g., are configured to provide for performance of a function, method, or operation) at least when one of the one or more of the distinct processors is capable of performing operations embodying the function, method, or operation. Processors 502 can perform operations embodying the function, method, or operation by, for example, executing code (e.g., interpreting scripts) stored on memory 504 and/or trafficking data through one or more ASICs. Processors 502, and thus processing system 500, can be configured to perform, automatically, any and all functions, methods, and operations disclosed herein. Therefore, processing system 500 can be configured to implement any of (e.g., all of) the protocols, devices, mechanisms, systems, and methods described herein.

For example, when the present disclosure states that a method or device performs task “X” (or that task “X” is performed), such a statement should be understood to disclose that processing system 500 can be configured to perform task “X”. Processing system 500 is configured to perform a function, method, or operation at least when processors 502 are configured to do the same.

Memory 504 can include volatile memory, non-volatile memory, and any other medium capable of storing data. Each of the volatile memory, non-volatile memory, and any other type of memory can include multiple different memory devices, located at multiple distinct locations and each having a different structure. Memory 504 can include remotely hosted (e.g., cloud) storage.

Examples of memory 504 include a non-transitory computer-readable media such as RAM, ROM, flash memory, EEPROM, any kind of optical storage disk such as a DVD, a Blu-Ray® disc, magnetic storage, holographic storage, a HDD, a SSD, any medium that can be used to store program code in the form of instructions or data structures, and the like. Any and all of the methods, functions, and operations described herein can be fully embodied in the form of tangible and/or non-transitory machine-readable code (e.g., interpretable scripts) saved in memory 504.

Input-output devices 506 can include any component for trafficking data such as ports, antennas (i.e., transceivers), printed conductive paths, and the like. Input-output devices 506 can enable wired communication via USB®, DisplayPort®, HDMI®, Ethernet, and the like. Input-output devices 506 can enable electronic, optical, magnetic, and holographic, communication with suitable memory 506. Input-output devices 506 can enable wireless communication via WiFi®, Bluetooth®, cellular (e.g., LTE®, CDMA®, GSM®, WiMax®, NFC®), GPS, and the like. Input-output devices 506 can include wired and/or wireless communication pathways.

Sensors 508 can capture physical measurements of environment and report the same to processors 502. User interface 510 can include displays, physical buttons, speakers, microphones, keyboards, and the like. Actuators 512 can enable processors 502 to control mechanical forces.

Processing system 500 can be distributed. For example, some components of processing system 500 can reside in a remote hosted network service (e.g., a cloud computing environment) while other components of processing system 500 can reside in a local computing system. Processing system 500 can have a modular design where certain modules include a plurality of the features/functions shown in FIG. 5. For example, I/O modules can include volatile memory and one or more processors. As another example, individual processor modules can include read-only-memory and/or local caches

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A computer-implemented method for predicting pathogen evolution, the computer-implemented method comprising:
computing a mutation trajectory of an input sample of a pathogen to be simulated by iteratively determining an update to a vector of changes based on a gradient that is computed with respect to the input sample using a loss associated with a prediction output of a trained differentiable surrogate model; and
reconstructing changes to the input sample based on the iterative updates to obtain a predicted pathway of the pathogen evolution.

2. The computer-implemented method according to claim 1, wherein the updates are determined iteratively until a stop criteria is met, after which the step of reconstructing the changes to the input sample is performed.

3. The computer-implemented method according to claim 2, wherein the stop criteria is based on a predetermined number of iterations or changes to the input sample, or is based on the prediction output of the surrogate model.

4. The computer-implemented method according to claim 1, wherein the surrogate model comprises an encoder, a decoder and a classifier, and is trained by:
inputting training samples and corresponding labels as inputs to the encoder that converts the inputs into latent representations;
inputting the latent representations into the decoder that reconstructs the inputs;
determining a reconstruction loss associated with the reconstructed inputs;
inputting the latent representations into the classifier that provides a classification of each of the latent representations to fit the labels;
determining a classification loss associated with the classifications; and
training the surrogate model using the reconstruction loss and the classification loss.

5. The computer-implemented method according to claim 1, wherein, at each iteration, the update to the vector of changes is added to a latent representation of the input sample and used for a subsequent prediction of the surrogate model, from which a subsequent loss is determined for determining a subsequent gradient for a subsequent update.

6. The computer-implemented method according to claim 5, wherein the changes to the input sample are reconstructed based on the latent representations used for the subsequent predictions of the surrogate model.

7. The computer-implemented method according to claim 1, wherein the updates to the vector of changes take into account an auxiliary loss term indicating whether the changes to the input sample are plausible mutations.

8. The computer-implemented method according to claim 1, further comprising storing the updates to the vector of changes, and tracking a path of the changes to the input sample based on the stored updates to the vector of changes.

9. The computer-implemented method according to claim 1, further comprising collecting a dataset that contains examples of viruses that infect humans, and viruses that infect animals, and training the surrogate model using the dataset as training data to at least classify whether a virus would infect a human or an animal as the prediction.

10. The computer-implemented method according to claim 1, further comprising collecting a dataset that contains genome sequences on viruses, labels that distinguish between animal and human version of the viruses and/or fitness information on the viruses, and training the surrogate model using the dataset as training data to predict viral fitness, wherein the loss is a regression loss, and wherein changes to the input sample identify antigen targets.

11. The computer-implemented method according to claim 1, further comprising designing a vaccine based on the predicted pathway of the pathogen evolution.

12. The computer-implemented method according to claim 1, wherein the changes to the input sample are used to rank potential variants of the pathogen, and/or to determine convergently evolving features and/or patterns in a genome of the pathogen that make humans susceptible to the pathogen.

13. The computer-implemented method according to claim 1, further comprising collecting a dataset that contains annotated genome sequences of bacterial pathogens and labels distinguishing susceptible and antibiotic-resistant strains of the bacterial pathogens, and training the surrogate model using the dataset as training data, wherein the changes to the input sample indicate genome regions and homologous proteins susceptible to development of antibiotic resistance.

14. A computer system for predicting pathogen evolution, the computer system comprising one or more hardware processors which, alone or in combination, are configured to provide for execution of the following steps:
computing a mutation trajectory of an input sample of a pathogen to be simulated by iteratively determining an update to a vector of changes based on a gradient that is computed with respect to the input sample using a loss associated with a prediction output of a trained differentiable surrogate model; and
reconstructing changes to the input sample based on the iterative updates to obtain a predicted pathway of the pathogen evolution

15. A tangible, non-transitory computer-readable medium having instructions thereon which, upon being executed by one or more processors, provide for predicting pathogen evolution by execution of the following steps:
computing a mutation trajectory of an input sample of a pathogen to be simulated by iteratively determining an update to a vector of changes based on a gradient that is computed with respect to the input sample using a loss associated with a prediction output of a trained differentiable surrogate model; and
reconstructing changes to the input sample based on the iterative updates to obtain a predicted pathway of the pathogen evolution.
